SEQUENCE LISTING (12 sequences)

SEQ ID NO: 1 - DNA, 22 bp, Artificial Sequence
  GAPDH, Forward qPCR primer
ccatgagaagtatgacaacagc

SEQ ID NO: 2 - DNA, 19 bp, Artificial Sequence
  GAPDH, Reverse qPCR primer
ttctagacggcaggtcagg

SEQ ID NO: 3 - DNA, 20 bp, Artificial Sequence
  OCT4, Forward qPCR primer
gagaggcaacctggagaatt

SEQ ID NO: 4 - DNA, 20 bp, Artificial Sequence
  OCT4, Reverse qPCR primer
ccagaggaaaggacactggt

SEQ ID NO: 5 - DNA, 20 bp, Artificial Sequence
  ZSCAN10, Forward qPCR primer
ccacagccccaagaaggaat

SEQ ID NO: 6 - DNA, 20 bp, Artificial Sequence
  ZSCAN10, Reverse qPCR primer
ctggggttctgcttccgaat

SEQ ID NO: 7 - DNA, 20 bp, Artificial Sequence
  DPPA5, Forward qPCR primer
ctgaaagccattttcggccc

SEQ ID NO: 8 - DNA, 20 bp, Artificial Sequence
  DPPA5, Reverse qPCR primer
gcttcggcaagtttgagcat

SEQ ID NO: 9 - DNA, 21 bp, Artificial Sequence
  ZSCAN10, Forward (external) Nested PCR primer
cttcctcgcagcagatcctaa

SEQ ID NO: 10 - DNA, 20 bp, Artificial Sequence
  ZSCAN10, Reverse (external) Nested PCR primer
gctcctgatctcgggaactc

SEQ ID NO: 11 - DNA, 21 bp, Artificial Sequence
  ZSCAN10, Forward (internal) Nested PCR primer
cccacctgaccattccttctt

SEQ ID NO: 12 - DNA, 20 bp, Artificial Sequence
  ZSCAN10, Reverse (internal) Nested PCR primer
aggcaattccttcttggggc

(19) United States
(12) Patent Application Publication
(10) Pub. No.: US 2023/0340595 A1
(43) Pub. Date: Oct. 26, 2023

(54) USE OF PLURIPOTENT MARKERS TO DETECT CONTAMINATING RESIDUAL UNDIFFERENTIATED PLURIPOTENT STEM CELLS

(71) Applicant: Novo Nordisk A/S, Bagsvaerd (DK)

(72) Inventors: Juan Carlos Villaescusa Ramirez, Copenhagen S (DK); Nicolaj Stroeyer Christophersen, Virum (DK); Fabian Vinzenz Roske, Farum (DK); Paschalis Efstathopoulos, Copenhagen (DK)

(21) Appl. No.: 17/908,750

(22) PCT Filed: Mar. 1, 2021
(86) PCT No.: PCT/EP2021/055017

§ 371 Date: Sep. 1, 2022

(30) Foreign Application Priority Data
Mar. 2, 2020 (EP) 20160339.6

(51) Int. Cl. C12Q 1/6881; C12N 5/0735

(52) U.S. Cl. CPC C12Q 1/6881; C12N 5/0606; C12Q 2600/158; C12N 2506/02

(57) ABSTRACT

The present invention relates to a method of screening a cell population for contaminating residual undifferentiated stem cells by detecting the expression of one or more markers in the cell population, which expression is effectively silenced as PSCs are differentiated into specialized cells of either of the three germ layers.

### CROSS-REFERENCE TO RELATED APPLICATIONS

This application is a 35 U.S.C. § 371 National Stage application of International Application PCT/EP2021/055017 (WO 2021175768), filed Mar. 1, 2021, which claims priority to European Patent Application 20160339.6, filed Mar. 2, 2020; the contents of which are incorporated herein by reference.

### TECHNICAL FIELD

The present invention relates generally to the field of stem cells, such as human embryonic stem cells. Methods are provided for detecting pluripotent stem cells (PSCs) in an in vitro cell population of differentiated cells derived from PSCs.

### BACKGROUND

The use of stem cells in medicine is being intensively pursued with the prospect of alleviating, potentially reversing and/or curing conditions for which only limited or no treatment is available today. The stem cell products for such treatment may be derived from human PSCs such as but not limited to embryonic stem cells or induced PSCs. Human PSCs are largely undifferentiated cells with the potential to proliferate and differentiate into a number of more specialized cells of the human body. Established methods for obtaining stem cell-derived differentiated cells for the treatment of various conditions have already been developed, including protocols for providing ventral midbrain dopaminergic cells, retinal pigment epithelium (RPE) cells, neural retina cells, pancreatic islets containing beta cells, and cardiomyocytes. Such protocols, however, are typically not completely efficient and often result in a cell population comprising the intended cells as well as other cell types that may or may not be suitable of use in a final medicinal product. Furthermore, for some treatments it may not be viable to administer the fully differentiated or matured cells. In these cases, the differentiation of the cells is not fully completed in vitro as the cells are then intended to further mature in vivo after administration into the patient. Depending on the level of maturity, the medicinal product may still contain some small fraction of cells in a mitotic stage with high capacity to proliferate. A stem cell-derived population wherein the differentiated cells have not fully matured may comprise a mixture of cells at various developmental stages. Even for cell populations derived according to a differentiation protocol for which fully matured cells are intended a subset of the cells may still be at a mitotic stage or may even be pluripotent.

When aiming to provide a patient-safe treatment it is undesirable if a stem cell-derived product for administration comprises PSCs and/or PSC-like cells with the inherent potential to proliferate and develop into almost any cell type. The major concern being the risk of uncontrollable proliferation of the cells, which could potentially develop into a teratoma or malignant tumor or a cancer-like state.

Continued development of the differentiation protocols as well as optional purification processes may result in a highly pure cell population. Even still, to ensure patient safety and to comply with regulations by health authorities a quality control of stem cell-derived products is required for verifying that a product is not contaminated with residual undifferentiated cells, in particular PSCs or PSC-like cells.

Several genetic markers (and their coded proteins) are well characterized in human PSC. As PSCs are differentiated into a specific germ layer and further into a more specialized cell type the gene expression of the cell will change. This suggests for using genetic markers to establish the type and maturity of the cell. Multiple markers identifying human PSCs are known. Depending on the cell type into which the PSC is differentiated many markers expressed at the pluripotent stage will to some extent become quiescent. This can be utilized in identifying PSCs in a cell population of differentiated cells. However, the timing and extent of the expression of pluripotent markers being silenced differ for different cell types making a generic method of detecting the PSCs difficult. Furthermore, a pluripotent cell is defined by the co-expression of different pluripotent markers, and the use of only one specific gene as a marker to assess pluripotency might provide false positive results, which may lead to discard batches of differentiated cells, which do actually not contain residual PSCs. The present inventors have found that well-established markers for pluripotency such as OCT4 (POU5F1), SOX2, NANOG, and LIN28A may also be expressed in cells that have differentiated and lost pluripotency.

It is therefore an object of the present invention to overcome the aforementioned challenges, in particular it is an object to provide a robust method for detecting contaminating residual undifferentiated PSCs in a cell product, wherein the method offers a low risk of resulting in false positives. It is further an object that the provided method may be applied across various protocols for obtaining different stem cell-derived products, i.e. applicable to multiple cell types spanning across the three germ layers and at various stages of maturity.

### SUMMARY

The objects as outlined above are achieved by the aspects of the present invention. In addition, the present invention may also solve further problems, which will be apparent from the disclosure of the exemplary embodiments.

In a first aspect of the present invention is provided a method of screening a cell population for contaminating residual undifferentiated stem cells comprising the step of detecting the expression of a marker in the cell population, wherein the marker is selected from ZSCAN10, DPPA5, and FOXD3. The present inventors have found that the expression of these particular markers is efficiently silenced as the stem cells lose pluripotency. This holds true for the differentiation of the PSCs into a variety of different cell types across the three different germ layers (endoderm, mesoderm, ectoderm), which makes the method highly suitable for generic testing of stem cell derived products. The particular markers are silences to a level that results in a very low number of false positive. In an embodiment, the expression of the marker ZSCAN10 is detected. The present inventors have demonstrated that this marker is highly downregulated in a variety of differentiated cells. Accordingly, the screening of ZSCAN10 marker alone is highly suitable and sufficient to identify residual undifferentiated cells in a drug product.

In another aspect is provided a cell population comprising differentiated cells derived from PSCs, wherein the cell population is devoid of cells expressing one or more of the markers selected from ZSCAN10, DPPA5, and FOXD3. In a particular embodiment, the cell population is devoid of cells expressing ZSCAN10.

### BRIEF DESCRIPTION OF DRAWINGS

FIG. 1 shows qPCR analyses for various pluripotent markers, specifically fold difference in hESC relative to hESC-derived RPE cells.

FIG. 2 shows Ct values for ZSCAN10, DPPA5 and OCT4.

FIG. 3 shows bands for RPE samples for ZSCAN10, OCT4, and DPPA5 when the product was run in an agarose gel after the end of 40 cycles of the qPCR reaction.

FIG. 4 shows qPCR analyses of ZSCAN10 expression on hESC spiked-in samples. (A) and (B) show Ct values in relation to fraction of spiked-in hESC in hESC-derived RPE cells.

FIG. 5 shows qPCR analyses of ZSCAN10 expression in two different RPE batches.

FIG. 6 shows nested PCR for ZSCAN10 expression in hESC spiked-in samples. (A) shows first round of amplification of the nested PCR. (B) shows second round of PCR.

FIG. 7 shows expression fold-change of candidate markers between hESCs vs. BC-DS (left side) and hESCs vs. BC-DP (right side) as determined by qRT-PCR.

FIG. 8 shows expression fold-change of LIN28A and ZSCAN10 between hESCs vs. BC-DS (A) and hESCs vs. BC-DP (B) as determined by ddPCR.

FIG. 9 shows LIN28A and ZSCAN10 transcript copy numbers for BC-DS and BC-DP in ddPCR reactions with increasing cDNA input amounts.

FIG. 10 shows ZSCAN10 copies increase in a linear relationship with the fraction of hESCs spiked into BC-DP.

### DESCRIPTION

Unless otherwise stated, all technical and scientific terms used herein have the same meanings as commonly understood by one of ordinary skill in the art to which this invention belongs. The practice of the present invention employs, unless otherwise indicated, conventional methods of chemistry, biochemistry, biophysics, molecular biology, cell biology, genetics, immunology and pharmacology, known to those skilled in the art.

It is noted that all headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Throughout this application the terms “method” and “protocol” when referring to processes for differentiating cells may be used interchangeably. As used herein, “a” or “an” or “the” can mean one or more than one. Unless otherwise indicated in the specification, terms presented in singular form also include the plural situation. As used herein, “and/or” refers to and encompasses any and all possible combinations of one or more of the associated listed items, as well as the lack of combinations when interpreted in the alternative (“or”). Moreover, the present invention also contemplates that in some embodiments of the invention, any feature or combination of features set forth herein can be excluded or omitted.

In general and unless otherwise stated “day 0” refers to the initiation of the protocol, this be by for example but not limited to plating the stem cells or transferring the stem cells to an incubator or contacting the stem cells in their current cell culture medium with a compound prior to transfer of the stem cells. Typically, the initiation of the protocol will be by transferring undifferentiated stem cells to a different cell culture medium and/or container such as but not limited to by plating or incubating, and/or with the first contacting of the undifferentiated stem cells with a compound that affects the undifferentiated stem cells in such a way that a differentiation process is initiated.

Hereinafter, the methods according to the present invention are described in more detail by non-limiting embodiments and examples. A method is provided for screening a cell population for PSCs or PSC-like cells. By “pluripotent stem cell” (PSC) is to be understood an undifferentiated cell having differentiation potency and proliferative capacity (particularly self-renewal competence) but maintaining differentiation potency. The stem cell includes subpopulations such as PSC, multipotent stem cell, unipotent stem cell and the like according to the differentiation potency. PSC refers to a stem cell capable of being cultured in vitro and having a potency to differentiate into any cell lineage belonging to three germ layers (ectoderm, mesoderm, endoderm). The multipotent stem cell means a stem cell having a potency to differentiate into plural types of tissues or cells, though not all kinds. The unipotent stem cell means a stem cell having a potency to differentiate into a particular tissue or cell. A PSC can be induced from fertilized egg, clone embryo, germ stem cell, stem cell in a tissue, somatic cell and the like. Examples of the PSC include embryonic stem cell (ES cell), EG cell (embryonic germ cell), induced pluripotent stem cell (iPSC) and the like. Muse cell (Multi-lineage differentiating stress enduring cell) obtained from mesenchymal stem cell (MSC), and GS cell produced from reproductive cell (e.g., testis) are also encompassed in the PSC. iPSCs are a type of PSC that can be generated directly from adult cells. By the introduction of products of specific sets of pluripotency-associated genes adult cells can be converted into PSCs. Embryonic stem cells can be produced by culturing cells from a blastomere or the inner cell mass of a blastocyst. Such cells can be obtained without the destruction of the embryo. Embryonic stem cells are available from given organizations and are also commercially available.

As used herein, beta-like cells before and after the purification step will be referred to as beta-cell drug substance (BC-DS) and beta-cell drug product (BC-DP), respectively.

In a first aspect of the present invention is provided a method of screening a cell population for contaminating residual undifferentiated stem cells comprising the step of detecting the expression of a marker in the cell population, wherein the marker is selected from ZSCAN10, DPPA5, and FOXD3.

As used herein, the term “cell population” refers to a defined group of cells, which may be in vitro or in vivo. Typically, the group of cells will be isolated in vitro in a container. In a preferred embodiment, the method according to the present invention is carried out in vitro. In an embodiment, the in vitro container is a suitable substrate such as a microwell.

As used herein, the term “contaminating residual undifferentiated stem cells” refers to a subpopulation of PSCs in a cell population having been subject to a differentiation protocol intended to differentiate the cell population into differentiated cells without pluripotent properties.

As used herein, the term “screening” refers to the action of examining the cell population for the presence of one or more cells having a certain genotype or phenotype, such as pluripotency. The genotype and phenotype may be established based on the expression of markers.

As used herein, the term “marker” refers to a naturally occurring identifiable expression made by a cell, which can be correlated with certain properties of the cell. In a preferred embodiment the marker is a genetic or proteomic expression, which can be detected and correlated with the identity of the cell. The markers may be referred to by gene. This can readily be translated into the expression of the corresponding mRNA and proteins.

As used herein, the term “expression” in reference to a marker refers to the lack or presence in the cell of a molecule, which can be detected. In an embodiment, the expressed molecule is mRNA or a protein. Accordingly, in an embodiment the PSCs are detected, and optionally identified, on a transcriptomic and/or proteomic level. In an embodiment, the marker is the genetic expression of a gene, which can be correlated with pluripotency of a stem cell. The expression of the marker may be detected at any suitable level, such as at mRNA or protein level. A person skilled in the art will readily appreciate that a cell can be defined by the positive or negative expression of a marker, i.e. the properties and state of a cell may equally be correlated based on the expression of a certain marker as well as the lack thereof. When referring to specific markers the presence or lack of expression may be denoted with + (plus) or − (minus) signs, respectively.

As used herein, the term “detecting” in reference to expression means measuring a signal to establish the presence of contaminating residual undifferentiated stem cells in a cell population. “Detecting” according to the method does not imply that a positive signal must be obtained, which would not be the case if the cell population does not comprise any contaminating residual undifferentiated stem cells. Any suitable signal may be used to establish the presence of PSCs, such as by the emission of light from e.g. fluorescent molecules. Numerous techniques are readily available to detect and optionally identify markers in a cell population. In one embodiment, the cell population is screened using bulk RNA-seq (RNA sequencing) analysis. As used herein, the term “bulk” when referring to screening means analyzing the expression of a marker in a cell population not the individual cells.

As used herein, “LIN28A” refers to the gene denoted Lin-28 homolog A. This gene is a marker of undifferentiated human embryonic stem cells.

As used herein, “POU5F1” refers to the gene denoted POU domain, class 5, transcription factor 1. The gene may also be referred to as OCT4. This gene is a marker of undifferentiated human embryonic stem cells.

As used herein, “SOX2” refers to the gene denoted SRY (sex determining region Y)—box 2. This gene is a marker of undifferentiated human embryonic stem cells. However, SOX2 is also expressed throughout developing neural stem cells.

As used herein, “NANOG” may also refer to the gene denoted Homeobox Transcription Factor NANOG. This gene is a marker of undifferentiated human embryonic stem cells.

As used herein, “ZSCAN10” refers to the gene denoted “Zinc Finger And SCAN Domain Containing 10”. This gene has been reported to encode a transcriptional factor for regulation of PSCs. It is expressed in undifferentiated human and mouse embryonic stem cells, the inner cell mass of blastocysts and down regulated upon differentiation. ZSCAN10 is considered to maintain ESC pluripotency by interacting with the established pluripotency markers SOX2 and OCT4.

As used herein, “DPPA5” refers to the gene denoted “Developmental Pluripotency Associated 5”. This gene has been reported to encode a protein that may function in the control of cell pluripotency and early embryogenesis. Expression of this gene is therefore believed to be a specific marker for PSCs involved in the maintenance of embryonic stem cell pluripotency. It is believed to play an important role in human PSC self-renewal and cell reprogramming in feeder-free conditions.

As used herein, “FOXD3” refers to the gene denoted “Forkhead Box D3”. Multiple studies have suggested Foxd3 involvement in the transition from naive to primed PSCs in embryo development. Previously, FOXD3 was demonstrated to be required in maintaining pluripotency in mouse embryonic stem cells.

In one embodiment, the expression of two or more markers selected from ZSCAN10, DPPA5, and FOXD3 is detected. In another embodiment, the expression of the markers ZSCAN10 and DPPA5 is detected. In another embodiment, the expression of the markers ZSCAN10, DPPA5 and FOXD3 is detected. In a preferred embodiment, the expression of the marker ZSCAN10 is detected.

In one embodiment, the presence of contaminating residual undifferentiated stem cells in a cell population is established by the positive expression of either one of the markers ZSCAN10, DPPA5, or FOXD3 using bulk analysis of the cell population. In a further embodiment, the bulk analysis is by RNA-seq analysis.

In an embodiment, the cell population comprises differentiated cells derived from PSCs. As used herein, the term “differentiated cells” in respect to stem cells refers to PSCs, which have undergone a process wherein the cells have progressed from an undifferentiated state to a specific differentiated state, i.e. from an immature state to a less immature state or to a mature state. Changes in cell interaction and maturation occur as cells lose markers of undifferentiated cells or gain markers of differentiated cells. Loss or gain of a single marker can indicate that a cell has matured or fully differentiated. “Differentiated cells” are therefore considered to be cells which have previously been classified as PSCs but allowed to differentiate into the cell type of a certain germ layer.

It follows that in an embodiment, the method comprises an initial step of differentiating PSCs into a cell population of differentiated cells derived from the PSCs. One of ordinary skill in the art will readily appreciate that as used herein the term “differentiating” refers to subjecting the PSCs to a method which progresses the cells from an undifferentiated state to a differentiated state. Typically, a step of differentiating PSCs involves culturing the cells under certain conditions and/or contacting the cells with certain factors.

In an embodiment, the PSCs are human PSCs. In a further embodiment, the PSCs are human embryonic stem cells.

In an embodiment, the differentiated cells are selected from ventral midbrain dopaminergic cells, retinal pigment epithelium (RPE) cells, neural retina cells, pancreatic islets containing beta cells, and cardiomyocytes. A person skilled in the art will recognize suitable methods for differentiating PSCs into the aforementioned cell types. For example, a protocol for obtaining ventral midbrain dopaminergic cells is disclosed in patent application WO 2016/162747. Depending on the level of maturation the ventral midbrain dopaminergic cells may express of one or more of the markers FOXA2, LMX1B, OTX2, EN1, PITX3, and TH. A protocol for obtaining RPE cells is disclosed by Osakada et al (J Cell Sci. 2009 Sep 1;122(Pt 17):3169-79. doi: 10.1242/jcs.050393. Epub 2009 Aug 11. “In vitro differentiation of retinal cells from human pluripotent stem cells by small-molecule induction”) or by Kuroda et al. (Stem Cell Res. 2019 Aug;39:101514. doi: 10.1016/j.scr.2019.101514. Epub 2019 Jul 25. “Robust induction of retinal pigment epithelium cells from human induced pluripotent stem cells by inhibiting FGF/MAPK signaling”). Depending on the level of maturation the RPE cells may express one or more of the markers MITF and RPE65. A protocol for obtaining neural retina progenitor cells is disclosed by Xie et al (PLoS One. 2014 Nov 17;9(11):e112175. doi: 10.1371/journal.pone.0112175. eCollection 2014. “Differentiation of retinal ganglion cells and photoreceptor precursors from mouse induced pluripotent stem cells carrying an Atoh7/Math5 lineage reporter”) or patent application WO 2019/078781. Depending on the level of maturation the neural retina cells may express the marker OTX2. A protocol for obtaining pancreatic islets containing beta cells is disclosed by Robert et al. (Stem Cell Reports. 2018 Mar 13;10(3):739-750. doi: 10.1016/j.stemcr.2018.01.040. Epub 2018 Mar 1. “Functional Beta Cell Mass from Device-Encapsulated hESC-Derived Pancreatic Endoderm Achieving Metabolic Control”) or by Bukys et al. (J Stem Cell Transplant Biol. 2016 Sep 21;2(1). doi: 10.19104/jorm.2017.109. “Xeno-Transplantation of macro-encapsulated islets and Pluripotent Stem Cell-Derived Pancreatic Progenitors without Immunosuppression”) or patent application WO 2017/144695. Beta cells may be defined by the expression of the markers NKX6. 1+/INS+/GCG−. A protocol for obtaining cardiomyocytes is disclosed by Yap et al. (Cell Rep. 2019 Mar 19;26(12):3231-3245.e9. doi: 10.1016/j.celrep.2019.02.083. “In Vivo Generation of Post-infarct Human Cardiac Muscle by Laminin-Promoted Cardiovascular Progenitors”) or by Fernandes et al. (Stem Cell Reports. 2015 Nov 10;5(5):753-762. doi: 10.1016/j.stemcr.2015.09.011. “Comparison of Human Embryonic Stem Cell-Derived Cardiomyocytes, Cardiovascular Progenitors, and Bone Marrow Mononuclear Cells for Cardiac Repair”).

The present inventors analyzed cell populations of RPE cells, neural retina progenitor cells, ventral midbrain dopaminergic progenitor cells, pancreatic islets containing beta cells, and cardiomyocytes, respectively, using single cell RNA-seq. None of the cell populations contained cells expressing the markers ZSCAN10, DPPA5 and FOXD3. In comparison analysis of cell populations of PCS identified cells expressing these markers.

In one embodiment, the cell population is in vitro. Most commonly, the cell population for screening will be an in vitro stem cell-derived product of differentiated cells intended for therapy. In one embodiment, the cell population is provided from a biopsy. Such biopsy may be obtained directly from a patient and analyzed in vitro to screen for PSCs.

In an embodiment, the method comprises the step of identifying residual PSCs or PSC-like cells in the cell population. As used herein, the term “PSC-like cells” means cells that have lost pluripotency but are still sharing some characteristics with PSCs such as some gene expression, capacity to proliferate or any other feature similar to PSCs. By the term “identifying” is meant establishing or indicating a strong link between detecting the expression of certain markers in a cell population and a specific cell of that cell population. In an embodiment, residual PSCs or PSC-like cells are detected, and optionally identified, by single cell sequencing. In one embodiment, the cell population is screened using fluorescence-activated cell sorting (FACS).

In another aspect is provided a cell population comprising differentiated cells derived from PSCs, wherein the cell population is devoid of cells expressing one or more of the markers selected from ZSCAN10, DPPA5 and FOXD3. In a further embodiment, the cell population is devoid of cells expressing ZSCAN10.

In an embodiment, the cell population has a detection value of the expression of one or more of the markers ZSCAN10, DPPA5 and FOXD3 below 0.1, 0.01, or 0.001% of hPSC mixed in the differentiated cells compared to a spike-in reference cell population. In another embodiment, the cell population has a detection value of the expression of one or more of the markers ZSCAN10 below 0.1, 0.01, or 0.001% of hPSCs mixed in the differentiated cells compared to a spike-in reference cell population.

As used herein, the term “devoid” is defined by the negative detection of one or more of the expression markers selected from ZSCAN10, DPPA5, and FOXD3. In an embodiment, the detection method is according to Example 1. In a preferred embodiment, the cell population has been screened according to the method of the first aspect of the present invention.

### Particular Embodiments

The aspects of the present invention are now further described by the following non-limiting embodiments:
1. A method of screening a cell population for contaminating residual undifferentiated stem cells comprising the step of detecting the expression of a marker in the cell population, wherein the marker is selected from ZSCAN10, DPPA5, and FOXD3.
2. The method according to the preceding embodiment, wherein the expression of two or more markers selected from ZSCAN10, DPPA5, and FOXD3 is detected.
3. The method according to any one of the preceding embodiments, wherein the expression of the markers ZSCAN10 and DPPA5 is detected.
4. The method according to any one of the preceding embodiments, wherein the expression of the markers ZSCAN10, DPPA5, and FOXD3 is detected.
5. The method according to any one of the preceding embodiments, wherein the expression of the marker ZSCAN10 is detected.
6. The method according to any one of the preceding embodiments, wherein the cell population comprises differentiated cells derived from PSCs.
7 The method according to embodiment 6, wherein the PSCs are human PSCs.
8. The method according to embodiment 7, wherein the PSCs are human embryonic stem cells.
9. The method according to any one of the preceding embodiments, comprising the initial step of differentiating PSCs into a cell population of differentiated cells derived from the PSCs.
10. The method according to any one of embodiments 6 to 9, wherein the differentiated cells are selected from ventral midbrain dopaminergic cells, retinal pigment epithelium (RPE) cells, neural retina cells, pancreatic islets containing beta cells, and cardiomyocytes.
11. The method according to any one of the preceding embodiments, wherein the cell population is in vitro.
12. The method according to any one of the preceding embodiments, wherein the cell population is provided from a biopsy.
13. The method according to any one of the previous embodiments, comprising the step of identifying residual PSCs or PSC-like cells in the cell population.
14. The method according to any one of the preceding embodiments, wherein residual PSCs or PSC-like cells are detected, and optionally identified, on a transcriptomic and/or proteomic level.
15. The method according to any one of the preceding embodiments, comprising a step of amplifying cDNA prior to the step of detecting the marker.
16. The method according to embodiment 15, wherein the cDNA (complementary DNA,

DNA synthesized from a single-stranded RNA) is amplified using RT-PCR, qPCR, or ddPCR, or a combination thereof.
17. The method according to any one of the previous embodiments, wherein the cell population is screened using fluorescence-activated cell sorting (FACS).
18. The method according to any one of the previous embodiments, wherein the cell population is screened using bulk analysis.
19. The method according to embodiment 18, wherein the bulk analysis is by RNA-seq.
20. The method according to any one of the previous embodiments, wherein the cell population is screened by single cell analysis.
21. The method according to embodiment 20, wherein the single cell analysis is by single cell RNA sequencing.
22. A cell population comprising differentiated cells derived from PSCs, wherein the cell population is devoid of cells expressing one or more of the markers selected from ZSCAN10, DPPA5, and FOXD3.
23. The cell population according to embodiment 22, wherein the population is devoid of cells expressing ZSCAN10.
24. The cell population according to any one of embodiments 22 and 23, wherein the cell population has been screened according to the method of any one of the embodiments 1 to 21.
25. The cell population according to any one of embodiments 22 to 24, wherein the PSCs are human PSCs.

### EXAMPLES

The following are non-limiting examples of protocols for carrying out the invention.

### Example 1: Single Cell RNA Sequencing (scRNAseq) Analysis

Populations of undifferentiated (hESCs) and differentiated cells were analyzed by single cell RNA-seq. Table 1 shows the number of cells in each population expressing specific genes. Single cell sequencing data was pre-processed and mapped to the human genome using the cellranger software provided by 10× and the automatically detected cells are subsequently filtered to remove cells that are most likely dead cells (low gene or UMI count, high mitochondrial gene content) or potential doublets (high gene/UMI count). Moreover, features (genes) that are detected (with a count>0) in less than 3 cells were disregarded (removed) from the downstream analysis. Due to the low per cell sequencing depth in scRNAseq, a detection limit of 1 UMI for marker evaluations was used.

### Example 2: Single Cell RNA-seq of Undifferentiated and Differentiated Cells

All markers were detected in the populations of hPSCs. Furthermore, the common markers associated with pluripotency, i.e. LIN28A, POU5F1, SOX2, and NANOG were all detected in some cells of the differentiated cell populations. SOX2 is a known marker expressed in the ectodermal lineage, which is confirmed by the high number of cells expressing this marker for dopaminergic progenitors, neural retina and RPE progenitor cells. For the differentiated populations, however, the combined expression of POU5F1, SOX2 and NANOG was not detected in any cell, which indicates that no pluripotent cells were present. None of the markers DPPA5, ZSCAN10, or FOXD3 were detected in any cell of the differentiated cell populations.

TABLE 1
Cell count of genetic markers expressed,
analyzed using single cell RNA-seq analysis

| | | | | Pan- | | | |
|---|---|---|---|---|---|---|---|
| | | | | creatic | | | |
| Differen- | | | | progen- | Cardio- | hESC | hESC |
| tiation | mesDA | NR | RPE | itors | myocytes | 1 | 2 |
| LIN28A | 4074 | nd | nd | 155 | 1609 | 2139 | 1347 |
| POU5F1 | 49 | 6 | 2 | 52 | 13 | 2143 | 3062 |
| SOX2 | 8598 | 1080 | 1275 | 311 | 9 | 2133 | 3057 |
| NANOG | 358 | 26 | 14 | 268 | 28 | 1454 | 1714 |
| POU5F1/ | 0 | 0 | 0 | 0 | 0 | 1448 | 1710 |
| SOX2/ | | | | | | | |
| NANOG | | | | | | | |
| DPPA5 | 0 | 0 | 0 | 0 | 0 | 317 | 345 |
| ZSCAN10 | 0 | 0 | 0 | 0 | 0 | 1925 | 2064 |
| FOXD3 | 0 | 0 | 0 | 0 | 0 | 537 | 473 |
| Total | 9236 | 1784 | 2148 | 6140 | 2095 | 2143 | 3062 |
| number of | | | | | | | |
| cells | | | | | | | |
| analyzed | | | | | | | |

nd: not determined; mesDA: mesencephalic dopaminergic progenitor cells; NR: neural retina progenitor cells; RPE: retinal pigmented epithelial cells; hESC: Human embryonic cell lines 1 and 2.

### Example 3: Single Cell RNA-seq Analysis of Markers during Differentiation from hESCs into RPE Cells

Single cell analysis was performed on cells at different stages of a protocol differentiating hESCs into RPE cells. The results of Table 2 clearly indicate that markers DPPA5, ZSCAN10, and FOXD3 are efficiently silenced early in the differentiation process, whereas the markers POU5F1, SOX2 and NANOG are still expressed in some cells during the late stage of the protocol. At day 30 cells with the combined expression of POU5F1, SOX2 and NANOG stop being detectable. At the same timepoint each of the markers DPPA5, ZSCAN10, and FOXD3 are no longer detectable.

TABLE 2
Single cell RNA-seq analysis during differentiation of RPE cells

| RPE | | | | | | |
|---|---|---|---|---|---|---|
| Differen- | | | | | | Day |
| tiation | Day 0 | Day 6 | Day 30 | Day 60 | Day 90 | 120 |
| LIN28A | nd | 2444 | nd | nd | nd | 2139 |
| POU5F1 | 3929 | 817 | 3 | 6 | 9 | 2 |
| SOX2 | 3908 | 2421 | 2028 | 1841 | 1407 | 1275 |
| NANOG | 2928 | 43 | 32 | 7 | 14 | 14 |
| POU5F1/ | 2913 | 18 | 0 | 0 | 0 | 0 |
| SOX2/ | | | | | | |
| NANOG | | | | | | |
| DPPA5 | 326 | 0 | 0 | 0 | 0 | 0 |
| ZSCAN10 | 3140 | 415 | 0 | 0 | 0 | 0 |
| FOXD3 | 1144 | 0 | 0 | 0 | 0 | 0 |
| Total | 3929 | 2446 | 2408 | 2505 | 2512 | 2148 |
| number of | | | | | | |
| cells | | | | | | |
| analyzed | | | | | | |

nd: not determined

### Example 4: Single Cell RNA-seq Analysis of Markers during Differentiation from hESCs into Neural Retina Cells

Single cell analysis was performed on cells at different stages of a protocol differentiating hESCs into neural retina cells. The results of Table 3 clearly indicate that markers DPPA5, ZSCAN10, and FOXD3 are efficiently silenced early as the cells differentiate, whereas the markers POU5F1, SOX2 and NANOG are still expressed in some cells during the late stage of the protocol. At day 20 cells with the combined expression of POU5F1, SOX2 and NANOG stop being detectable. At the same timepoint each of the markers DPPA5, ZSCAN10, and FOXD3 are no longer detectable.

TABLE 3
Single cell RNA-seq analysis during differentiation
of neural retina cells

| Neural Retina Differentiation | Day 0 | Day 6 | Day 20 | Day 30 |
|---|---|---|---|---|
| LIN28A | 3061 | 3397 | 1594 | nd |
| POU5F1 | 3062 | 906 | 7 | 6 |
| SOX2 | 3057 | 3424 | 3976 | 1080 |
| NANOG | 1714 | 44 | 101 | 26 |
| POU5F1/LIN28A/NANOG | 1714 | 11 | 0 | nd |
| DPPA5 | 345 | 0 | 0 | 0 |
| ZSCAN10 | 2064 | 626 | 0 | 0 |
| FOXD3 | 473 | 0 | 0 | 0 |
| Total number of cells analyzed | 3062 | 3449 | 4568 | 1784 |

nd: not determined

### Example 5: Quantitative Real-Time PCR (qRT-PCR) to Detect Residual Human Pluripotent Stem Cells

In the traditional qRT-PCR (or qPCR for simplification), the amplification of a sequence is followed by emerging fluorescence during the PCR reaction (Higuchi et al., Biotechnology (N Y). 1992 Apr;10(4):413-7. doi: 10.1038/nbt0492-413). qPCR is usually conducted to quantify the absolute amount of a target sequence or to compare relative amounts of a target sequence between samples. This technique monitors the amplification of the target in real-time via a target-specific fluorescent signal emitted during amplification. We used qPCR analysis to compare the expression level of OCT4 to those of ZSCAN10 and DPPA5, in hESC, hESC-derived RPE cells (RPE), and in various fractions of spiked-in hESC into RPE cells.

TABLE 4
Primers used for qPCR.

| Gene | Primer | Sequence (from 5′ to 3′) |
|---|---|---|
| GAPDH | Forward | CCATGAGAAGTATGACAACAGC |
| | | (SEQ ID NO: 1) |
| | Reverse | TTCTAGACGGCAGGTCAGG |
| | | (SEQ ID NO: 2) |
| OCT4 | Forward | GAGAGGCAACCTGGAGAATT |
| | | (SEQ ID NO: 3) |
| | Reverse | CCAGAGGAAAGGACACTGGT |
| | | (SEQ ID NO: 4) |
| ZSCAN10 | Forward | CCACAGCCCCAAGAAGGAAT |
| | | (SEQ ID NO: 5) |
| | Reverse | CTGGGGTTCTGCTTCCGAAT |
| | | (SEQ ID NO: 6) |
| DPPA5 | Forward | CTGAAAGCCATTTTCGGCCC |
| | | (SEQ ID NO: 7) |
| | Reverse | GCTTCGGCAAGTTTGAGCAT |
| | | (SEQ ID NO: 8) |

Briefly, RNA was extracted from 1.5×10⁶ cells per condition using RNeasy mini kit (Qiagen, 74104) following manufacturer instructions including DNAse I treatment. 500 ng RNA was converted to cDNA using the SuperScript™ IV VILO™ Master Mix (Invitrogen, 11756050). cDNA was diluted 1:10 and 1 ul was used per reaction. Power SYBR™ Green PCR Master Mix (applied biosystems, 4367659) was used for the qPCR run in viia7 Real time PCR system. In qPCR, the threshold line is the level of detection or the point at which a reaction reaches a fluorescent intensity above background levels. The Ct (threshold cycle) is the intersection between an amplification curve and a threshold line (Bustin et al., Clin Chem. 2009 Apr;55(4):611-22. doi: 10.1373/clinchem.2008.112797). The fold change relative to RPE expression was calculated using the ddCt (delat-delta Ct) method and GAPDH expression (housekeeping gene) as an endogenous control. Briefly, for each sample the dCt was initially calculated by subtracting the Ct value for the gene of interest from the Ct value of GAPDH. Subsequently, since we wanted to normalize everything to the expression level of the RPE cells, the dCt of the RPE sample for each gene was subtracted from the dCt of each of the other samples in order to calculate the ddCt. Finally the fold change was calculated using the formula 2\hat{ }(-ddCt).

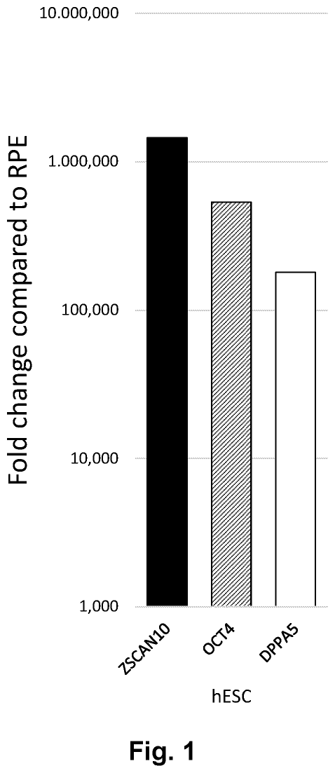

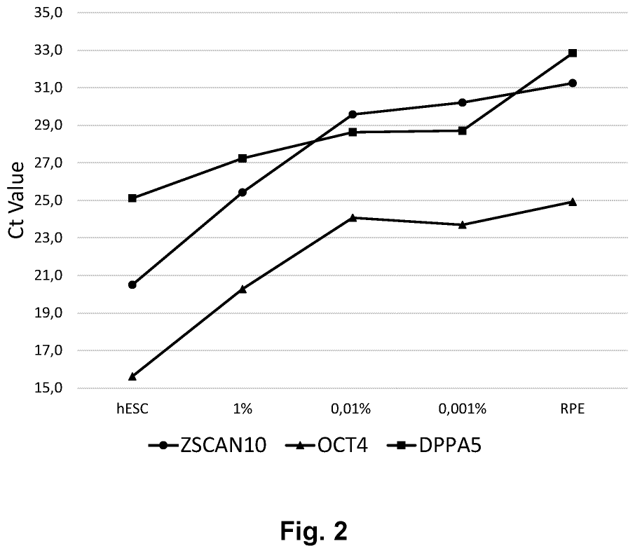

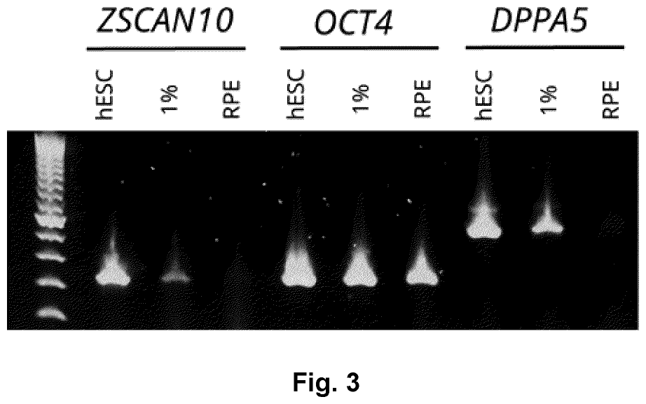

Among the genes tested, ZSCAN10 showed the highest fold difference in hESC relative to RPE cells (FIG. 1). In addition to this, RPE cells showed a Ct value over 30 for ZSCAN10 and DPPA5 (FIG. 2) and gave no visible bands when the product run in a gel after the end of 40 cycles of the qPCR reaction (FIG. 3). On the contrary, OCT4 seemed to be quite highly expressed in the RPE sample with a Ct value of 25 and gave a band in the gel that was of similar intensity of those samples that contained hESC. The above results support the idea that ZSCAN10 and potentially DPPA5 represent good candidates to be used with the nested PCR method for detection of pluripotent cells in the final RPE product.

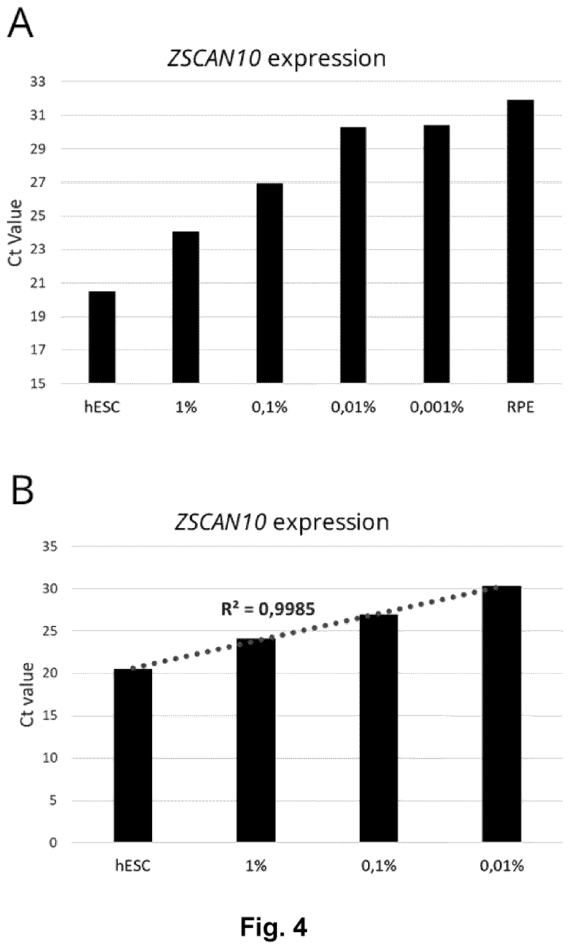

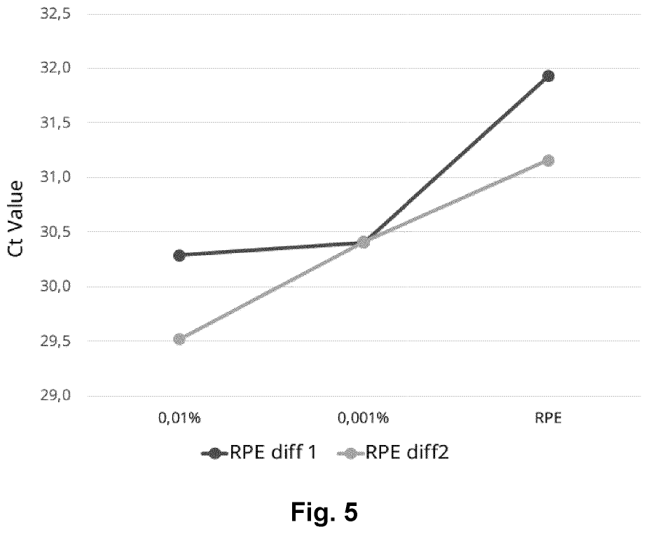

The qPCR analysis revealed a linear increase of Ct values in relation to the fraction of spiked-in hESC in RPE cells (FIG. 4A) up to the fraction of 0.01% (FIG. 4B). FIG. 5 shows the comparison between two different batches of hESC-derived RPE cells (RPE). qPCR analysis revealed an increase of Ct values for RPE cells in relation to the fraction of spiked-in hESC (0.01 and 0.001%), in two independent RPE differentiations (diff 1 and 2).

### Example 6: Nested RT-PCR for ZSCAN10 Marker to Detect Residual Human Pluripotent Stem Cells

Nested RT-PCR (or Nested PCR) involves the use of two pairs of primers in two successive reactions during which, the product of the first round is used as a template on the second round of amplification. The amplicon of the first reaction contains the target of the second reaction. The advantage of the nested PCR is the extensive amplification of the target sequence while reducing the chance (or appearance) of non-specific products (Green et al., Cold Spring Harb Protoc. 2019 Feb 1;2019(2). doi: 10.1101/pdb.prot095182). We developed a nested PCR assay based on ZSCAN10 marker as a simple and sensitive method to detect trace amounts of hESC cDNA, that would indicate the existence of residual pluripotent contaminants, in hESC-derived RPE (RPE) cells.

In order to use this technique for evaluating the existence of residual hESC in derived RPE cells, the marker of choice needs to be highly expressed in the undifferentiated cells and be completely absent in the differentiated ones.

Briefly, RNA was extracted from 1.5×10⁶ cells per condition using RNeasy mini kit (Qiagen, 74104) following manufacturer instructions including DNAse I treatment. 500 ng RNA was converted to cDNA using the SuperScript™ IV VILO™ Master Mix (Invitrogen, 11756050). RT minus samples are treated without the enzyme SuperScript, to detect possible genomic DNA contamination in the samples. For the first PCR, 1 ul of the 1:10 diluted cDNA was used per reaction. For the second PCR product the first PCR product was diluted 500 times and 1 ul was used per reaction. Template cDNA was amplified with Platinum II Hot start mastermix and 30 cycles in each round using the 2 step protocol according to manufacturer recommendations.

TABLE 5
Primers used for Nested PCR.

| Gene | Primer | Sequence (from 5′ to 3′) |
|---|---|---|
| ZSCAN10 | Forward (external) | CTTCCTCGCAGCAGATCCTAA |
| | | (SEQ ID NO: 9) |
| | Reverse (external) | GCTCCTGATCTCGGGAACTC |
| | | (SEQ ID NO: 10) |
| | Forward (internal) | CCCACCTGACCATTCCTTCTT |
| | | (SEQ ID NO: 11) |
| | Reverse (internal) | AGGCAATTCCTTCTTGGGGC |
| | | (SEQ ID NO: 12) |

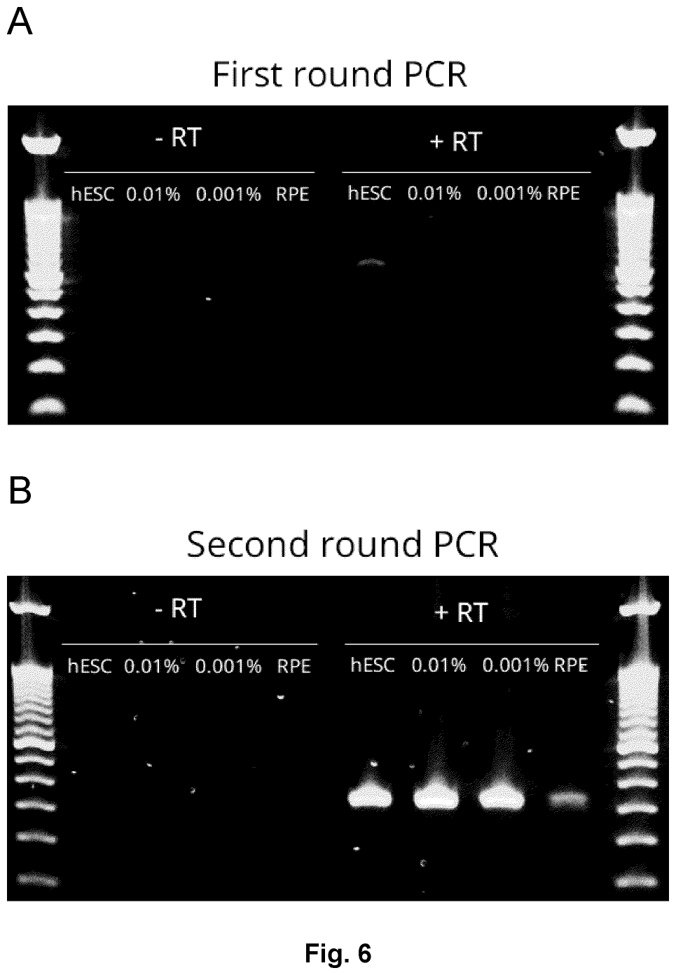

As it is shown in FIG. 6A, in the first round of amplification of the nested PCR using the external primers for ZSCAN10 gene we only get a detectable band in the hESCs sample. However, after the second round of PCR, using a fraction of the product produced in the first PCR for each sample and the pair of internal primers, we get saturated bands for all spike-in fractions tested (0.01 and 0.001%) but only a fade band in the RPE sample (FIG. 6B). These results indicate that there is substantially lower amount of ZSCAN10 cDNA in the RPE cells condition compared to the rest. The combination of one round of PCR with low number of cycles with a successive qPCR could be a way to increase sensitivity of detection and acquire more quantitative data.

### Example 7: Digital Droplet PCR for ZSCAN10 Marker to Detect Residual Human Pluripotent Stem Cells

We developed a digital droplet PCR (ddPCR) assay focused on the ZSCAN10 marker. In a digital droplet PCR reaction an endpoint PCR with 45 amplification cycles is done in physically separate water-oil emulsion droplets (Hindson et al., Anal Chem. 2011 Nov 15;83(22):8604-10. doi: 10.1021/ac202028g.). Compared to a traditional qRT-PCR, where the amplification of a sequence is followed by emerging fluorescence during the PCR reaction (Higuchi et al., Biotechnology (N Y). 1992 Apr;10(4):413-7. doi: 10.1038/nbt0492-413), ddPCR measures the distribution of positive and negative droplets after the reaction. Based on a Poisson distribution analysis, ddPCR determines the absolute concentration of an amplicon without the need for a standard curve. Besides this advantage, ddPCR has a higher sensitivity and accuracy compared to qRT-PCR, and there is no need to evaluate amplification efficiencies due to the endpoint readout.

For the same reasons, ddPCR has been used in the cell therapy field to evaluate the tumorigenicity risk in different cell types. Kuroda and colleagues were the first to adopt this technology for assessing their cardiomyocytes for residual hiPSCs using the pluripotency marker LIN28A (Kuroda et al., Regen Ther. 2015 Oct 27;2:17-23. doi: 10.1016/j.reth.2015.08.001). Piao and colleagues used ddPCR to assess their dopamine neurons for residual hESCs using the marker POU5F1, also known as OCT4 (Piao et al., Cell Stem Cell. 2021 Feb 4;28(2):217-229.e7. doi: 10.1016/j.stem.2021.01.004). Amongst other traditional pluripotency markers, we tested the suitability of these two markers to assess batches of insulin-containing pancreatic beta-like cells for residual hPSCs. The differentiation process towards these beta-like cell types takes between 20 and 30 days as it has been described in the literature by different groups (Pagliuca et al.,Cell. 2014 Oct 9;159(2):428-39. doi: 10.1016/j.cell.2014.09.040; Rezania et al., Nat Biotechnol. 2014 Nov;32(11):1121-33. doi: 10.1038/nbt.3033).

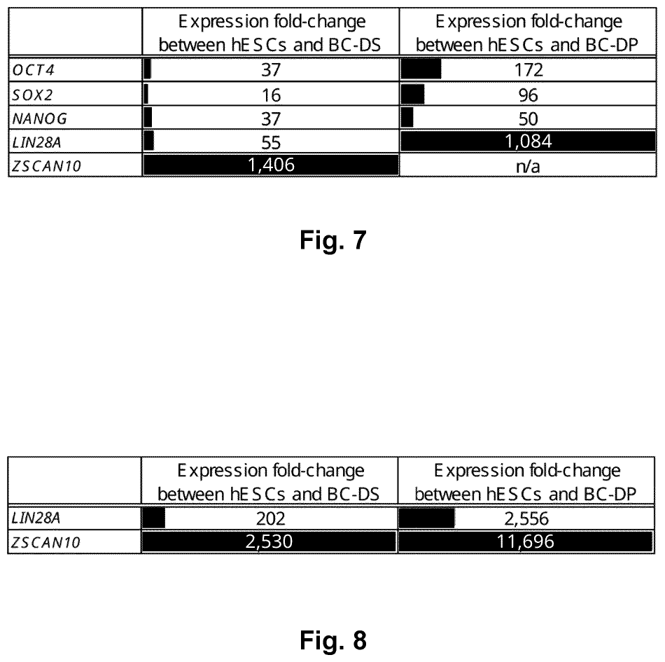

The most important parameter to evaluate the suitability of candidate markers for assessing the level of contaminating hESCs in a differentiated cell type is the fold-change of expression between hESCs and the differentiated cell type. For this purpose, we evaluated the expression levels of different markers in hESCs, BC-DS and BC-DP initially by qRT-PCR: The classical pluripotency markers OCT4, NANOG and SOX2 (Boyer et al., Cell. 2005 Sep 23;122(6):947-56. doi: 10.1016/j.cell.2005.08.020), the above mentioned marker LIN28A (Kuroda et al., Regen Ther. 2015 Oct 27;2:17-23. doi: 10.1016/j.reth.2015.08.001) and our newly discovered candidate markers ZSCAN10. The expression of these markers in all samples was normalized to the geometric mean of the widely used housekeeping genes ACTB and PPIA (Panina et al., Sci Rep. 2018 Jun 7;8(1):8716. doi: 10.1038/s41598-018-26707-8). For all samples a concentration could be calculated with the exception of ZSCAN10 in BC-DP, where the real-time PCR instrument could not detect a signal. The resulting expression values of all markers in hESCs were divided through the corresponding expression values in BC-DS and BC-DP to generate FIG. 7. The samples were analyzed on a BioRad CFX384 qRT-PCR instrument with probe-based PCR assays. The expression was normalized to the geometric mean of ACTB and PPIA. Note that no expression fold-change could be calculated for ZSCAN10 between hESCs and BC-DP, as the expression in BC-DP was below the LLOD (Lower Limit Of Detection) of the qRT-PCR instrument.

The left side of FIG. 7 shows that neither OCT4 nor LIN28A are suitable markers for evaluating hESC contamination levels in BC-DS, as the expression fold-change is too low. As an example, OCT4 with an expression fold-change of 37 can only be used to exclude the absence of hESCs in the BC-DS sample at a maximum sensitivity of 1 hESC in 37 BC-DS cells, when following the principles as laid out by Kuroda and colleagues for LIN28A (Kuroda et al., Regen Ther. 2015 Oct 27;2:17-23. doi: 10.1016/j.reth.2015.08.001). In contrast, ZSCAN10 is a much better markers with an expression fold-change of 1406. FIG. 7 furthermore shows that the expression fold-change between hESCs and BC-DP is, for all markers, larger than between hESCs and BC-DS. This observation is in alignment with the notion that the purification step between BC-DS and BC-DP eliminates proliferating off-target cell populations. In contrast to OCT4, LIN28A is a suitable candidate marker for evaluating hESC contaminants in BC-DP. Due to the observation that the expression of ZSCAN10 in BC-DP was not detectable by qRT-PCR, we hypothesized that the expression fold-change for ZSCAN10 is superior to that of LIN28A. To confirm this hypothesis, we analyzed hESCs, BC-DS and BC-DP by ddPCR that has a higher sensitivity than qRT-PCR (FIG. 8). The samples were analyzed on a BioRad qRT-PCR instrument with probe-based PCR assays. The expression was normalized to the geometric mean of ACTB and PPIA. Note that no expression fold-change could be calculated for ZSCAN10 between hESCs and BC-DP, as the expression in BC-DP was below the LLOD of the qRT-PCR instrument. This analysis confirms our hypothesis, as it shows that shows that the expression fold-change of ZSCAN10 is considerably larger than that of LIN28A. We therefore conclude that ZSCAN10 is a better choice for evaluating residual hESCs in both BC-DS and BC-DP than LIN28A. To our knowledge, there are at least three publications describing LIN28A as a pluripotency marker in the context of tumorigenicity assays (Artyuhov et al., Mol Biol Rep. 2019 Dec;46(6):6675-6683. doi: 10.1007/s11033-019-05100-2; Kuroda et al., PLoS One. 2012;7(5):e37342. doi: 10.1371/journal.pone.0037342; Kuroda et al., Regen Ther. 2015 Oct 27;2:17-23. doi: 10.1016/j.reth.2015.08.001), but none for ZSCAN10.

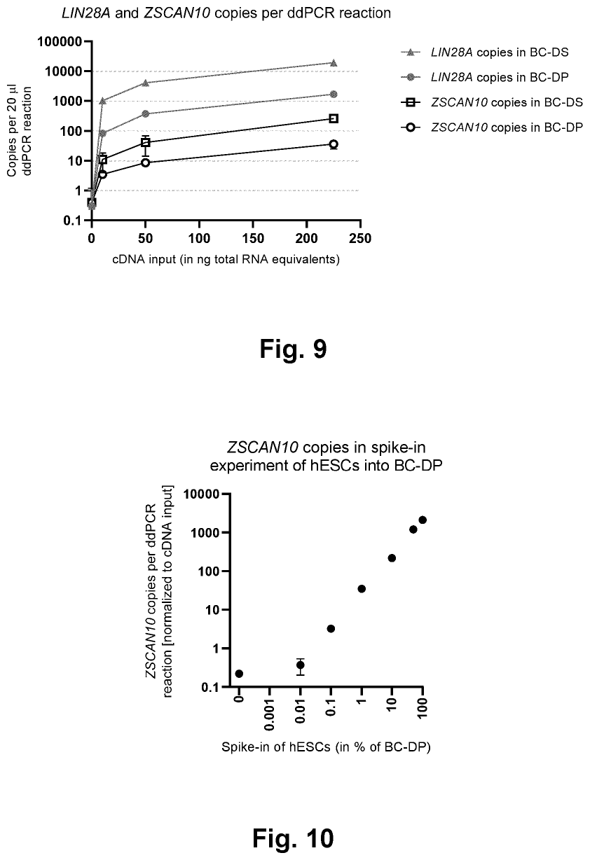

To confirm the suitability of ZSCAN10 as a candidate marker for assessing BC-DS and BC-DP, we increased the cDNA input for these samples from an equivalent of 50 ng total RNA per ddPCR reaction—a typical amount also used by Kuroda and colleagues (Kuroda et al., Regen Ther. 2015 Oct 27;2:17-23. doi: 10.1016/j.reth.2015.08.001)—to 225 ng (FIG. 9). The graph shows the absolute copy number of these transcripts per standard 20 μL ddPCR reaction in relation to the cDNA input described in equivalents of total RNA. This analysis shows that the absolute copy number of ZSCAN10 transcripts is lower in BC-DS and BC-DP compared to LIN28A transcript copy numbers. In addition to the expression fold-difference shown in FIG. 8, this further supports the suitability of ZSCAN10 as a novel marker for assessing residual hPSCs.

At last, we wanted to confirm the suitability of ZSCAN10 as a new marker in a spike-in experiment. For this purpose, we mixed defined cell numbers of hESCs into BC-DP, lysed the cells for RNA extraction, and executed ddPCR for ZSCAN10 reactions. FIG. 10 shows a nearly perfect linear relationship between ZSCAN10 copy numbers and the fraction of spiked-in hESCs. It should be noted that the y-axis shows ZSCAN10 values normalized to the amount of cDNA input. This normalization was necessary in order to fit the copy numbers into the acceptable dynamic range of the BioRad ddPCR setup used. We can confirm that ZSCAN10 can be used to detect contaminating hESCs to a LLOD of 0.01% (i.e. 1 hESC in 10,000 cells of BC-DP). We expect to further improve the sensitivity in two ways: (1) By increasing the RNA input, we will represent more cells in each reaction and (2) by using microfluidic technologies instead of serial dilutions, we will decrease the error in low-percentage spike-in samples and thus lower the LLOD.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A method of screening a cell population for contaminating residual undifferentiated stem cells comprising the step of detecting the expression of a marker in the cell population, wherein the marker is selected from ZSCAN10, DPPA5, and FOXD3.

2. The method according to claim 1, wherein the expression of two or more markers selected from ZSCAN10, DPPA5, and FOXD3 is detected.

3. The method according to claim 1, wherein the expression of the markers ZSCAN10 and DPPA5 is detected.

4. The method according to claim 1, wherein the expression of the markers ZSCAN10, DPPA5 and FOXD3 is detected.

5. The method according to claim 1, wherein the expression of marker ZSCAN10 is detected.

6. The method according to claim 1, wherein the cell population comprises differentiated cells derived from PSCs.

7. The method according to claim 1, wherein the cell population comprises differentiated cells selected from ventral midbrain dopaminergic cells, retinal pigment epithelium (RPE) cells, neural retina cells, beta cells, and cardiomyocytes.

8. The method according to claim 6, wherein the PSCs are human embryonic stem cells.

9. The method according to claim 1, wherein the cell population is in vitro.

10. The method according to claim 1, wherein the cell population is provided from a biopsy.

11. The method according to claim 1, wherein the cell population is screened using bulk analysis.

12. The method according to claim 11, wherein the bulk analysis is by RNA-seq.

13. The method according to claim 1, wherein the cell population is screened using qPCR, nested PCR, ddPCR, or a combination thereof.

14. A cell population comprising differentiated cells derived from PSCs, wherein the cell population is devoid of cells expressing one or more of the markers selected from ZSCAN10, DPPA5 and FOXD3.

15. The cell population according to claim 14, wherein the cell population has been screened for contaminating residual undifferentiated stem cells comprising the step of detecting the expression of a marker in the cell population, wherein the marker is selected from ZSCAN10, DPPA5, and FOXD3.
